(19) United States
(12) Patent Application Publication
(10) Pub. No.: US 2008/0077444 A1
(43) Pub. Date: Mar. 27, 2008

(54) SURGERY VALIDATION APPARATUS, SYSTEMS AND METHODS

(76) Inventor: Ronald R. MacLeod, Mississauga (CA)

Correspondence Address: HERMAN & MILLMAN, 425 UNIVERSITY AVENUE, SUITE 300, TORONTO, ON, M5G 1T6, US

(21) Appl. No.: 11/534,354

(22) Filed: Sep. 22, 2006

(51) Int. Cl. G06F 19/00; A61M 5/00

(52) U.S. Cl. 705/3; 604/189

(57) ABSTRACT

A surgical pause is a well intentioned pre-surgery procedure designed to reduce the likelihood that a patient may undergo an incorrect surgical procedure. However, the problem is that a surgical pause is often not taken as seriously as it should be and the surgical pause is performed in an ad hoc manner. Moreover, given the way in which the surgical pause is carried out, there is no way to enforce compliance with the spirit of the surgical pause and/or to check after the surgery that the surgical pause was performed with the requisite care and diligence by the health-care professionals in the operating theater. Many of the mistakes made in an operating theater can be attributed to not having a more systematic method for performing a surgical pause before a surgical procedure begins. In accordance with aspects of the invention apparatus, systems and methods are provided that may enable compliance to the intended spirit of the surgical pause and provide a record that a surgical pause was performed with the requisite care and diligence by the operating-room staff.

### FIELD OF THE INVENTION

The invention relates to pre-surgery patient care, and in particular to apparatus, systems and methods for enabling compliance to preferred practices before a surgical procedure begins.

### BACKGROUND OF THE INVENTION

Human error within an inpatient health-care facility can have devastating consequences. As such preferred practices are often codified as standard operating procedures that health-care professionals are mandated to follow. However, strict repetition of standard operating procedures by health-care professionals is difficult to enforce because many such procedures are thought to be common sense or overly simple to have been seriously codified as standard operating procedures. So while health-care professionals may be following the literal steps of the procedures, the spirit of those procedures is often treated with a cavalier attitude or even some disdain. Consequently, patient care may suffer and in extreme circumstances patients are victims of careless errors with devastating results.

One example of such a careless error occurs in an operating theater (i.e. operating room) just before a patient is operated on. Surgeons, anesthesiologists and nurses in the operating theater are typically required to perform a surgical pause, during which time the patient identity, the surgical procedure to be performed and the body part on which the surgical procedure is to be performed on is double-checked. That is, the surgical pause is designed to reduce the likelihood that a patient may undergo an incorrect surgical procedure. A problem is that the surgical pause is often not taken as seriously as it should be. In many cases, the health-care professionals involved assume that they are in the correct operating theater with the correct patient and are aware of what they are doing there. So after a brief repetition of the surgical procedure to be performed is spoken aloud, the doctors and nurses (and other specialists) proceed under the assumption that they are in the right place, at the right time, with the right patent patient performing the correct surgical procedure without further thought. A nurse typically notes in the respective patient chart that the surgical pause was carried out, but there is no guarantee that the spirit of the surgical pause was taken seriously.

Specific examples of such mistakes include, without limitation: amputating the wrong leg or arm; performing an eye surgery on the wrong eye; only learning that the necessary implants are not at hand or even in stock for a surgery to be performed after the surgery has been started; and amputating a leg instead of performing by-pass heart surgery. These are all real errors that have occurred in hospitals in North America. The consequences to the respective patients and even the health-care professionals were both physically and emotionally devastating.

Moreover, given that the current method of performing the surgical pause is ad hoc, there is no record kept that the surgical pause was actually performed and that the doctors, anesthesiologists and nurses followed the procedure as set out in the codified guidelines. In other words, there is no way to enforce compliance with the spirit of the surgical pause and/or check after the surgery that the surgical pause was performed with the requisite care and diligence by the health-care professionals in the operating theater.

### SUMMARY OF THE INVENTION

According to an aspect of an embodiment of the invention there is provided a surgery validation card comprising: a planar writing surface; at least one field printed on the planar writing surface having a checklist to be completed for verifying a respective identity of a patient in an operating theatre and a surgical procedure to be performed on the patient.

In some embodiments the surgery validation card is sufficiently sterilized so as to be suitable for use in a sterile field within an operating theatre.

In some embodiments the surgery validation card is removably integrated into a sleeve. In some more specific embodiments the surgery validation card is connected to the sleeve employing at least one of a perforated edge, a connecter and adhesive tape.

In some embodiments the machine-readable information storing information about the patient. In some more specific embodiments the machine-readable information includes at least one of a patient identifier, a surgical procedure identifier, a required equipment/implant listing, an allergy notice, a required image listing and antibiotic protocol reminder. In some other specific embodiments the machine-readable information is stored in at least one of a bar-code printed on the surgery validation card and a Radio Frequency Identification (RFID) tag included on the surgery validation card.

In some embodiments the surgery validation card further comprises a title field printed on the planar writing surface including a document identifier and a short list of instructions.

In some embodiments the surgery validation card further comprises a patient identification field, printed on the planar writing surface, including sub-fields for writing a respective patient name and corresponding patient identification number.

In some embodiments the surgery validation card further comprises a procedure identification field, printed on the planar writing surface, including sub-fields for writing a respective surgical procedure name, a site name, and side. In some more specific embodiments the surgery validation card further comprises a position verification field, printed on the planar writing surface, including a sub-field for writing a respective position description for the corresponding surgical procedure name.

In some embodiments the surgery validation card further comprises an imaging check field, printed on the planar writing surface, including a sub-field having a multiple-choice query to determine if any required imaging information is required for the surgical procedure.

In some embodiments the surgery validation card further comprises an equipment/implant check field, printed on the planar writing surface, including a sub-field having a multiple-choice query to determine if any required equipment/implants are required for the surgical procedure.

In some embodiments the surgery validation card further comprises an antibiotic protocol check field, printed on the planar writing surface, including a sub-field having a multiple-choice query to determine if a corresponding antibiotic protocol has been followed and documented for the surgical procedure.

In some embodiments the surgery validation card further comprises a signature field, printed on the planar writing surface, including at least one sub-field for a signature of one of the health-care professionals in an operating theatre present during a surgical pause.

According to an aspect of an embodiment of the invention there is provided a method of performing a surgical pause comprising: providing a sterile surgery validation card within a sterile field of an operating theatre; completing the surgery validation card before the initial incision or puncture of a surgical procedure to verify at least the identity of a patient and the surgical procedure to be performed; and, passing the completed surgery validation card outside the sterile field so that the completed surgery validation card can be placed into a corresponding patient chart.

According to some aspects the method further comprises: scanning machine-readable information on a surgery validation card and machine readable information on a patient bracelet; comparing the scanned machine-readable information from the surgery validation card and the patient bracelet; providing affirmative feedback if the surgery validation card and patient bracelet have the same scanned machine-readable information, and thereby by permitting the surgery validation card to be completed; and, providing dissenting feedback if the surgery validation card and patient bracelet do not have the same scanned machine-readable information.

According to some aspects providing the sterile surgery validation card includes placing the incomplete surgery validation card on one of the surgical instruments or on a portion of the patient.

According to some aspects the method passing the completed surgery validation card outside the sterile field includes physically separating the surgery validation card from a sleeve.

According to an aspect of an embodiment of the invention there is provided a surgery validation system comprising: a surgery validation card for verifying at least the identity of a patient and a surgical procedure to be performed, the surgery validation card including machine-readable information storing at least the identity of a patient and the name of the surgical procedure to be performed on the patient; a patient bracelet including machine-readable information storing at least the identity of a patient and the name of the surgical procedure to be performed on the patient; a machine-scanner for scanning machine-readable information and providing a scanned output; and, a workstation computer connectable to the machine-scanner for receiving the scanned output from the machine-scanner.

In some embodiments the machine-scanner and workstation computer are integral with one another.

In some embodiments the machine-readable information on the surgery validation card is stored in at least one of a bar-code printed on the surgery validation card and a Radio Frequency Identification (RFID) tag included on the surgery validation card.

In some embodiments, the machine-readable information on the patient bracelet is stored in at least one of a bar-code printed on the surgery validation card and a Radio Frequency Identification (RFID) tag included on the surgery validation card.

In some embodiments, the workstation computer includes a computer usable program code including program instructions for: receiving scanned machine-readable information from the surgery validation card and the patient bracelet; comparing the scanned machine-readable information from the surgery validation card and the patient bracelet; providing affirmative feedback if the surgery validation card and the patient bracelet have the same scanned machine-readable information; and, providing dissenting feedback if the surgery validation card and the patient bracelet do not have the same scanned machine-readable information.

In some embodiments the computer usable program code further includes program instructions for creating a record of the comparison.

In some embodiments the computer usable program code including program instructions for updating a database of records, wherein each record contains the results and a particular comparison.

Other aspects and features of the present invention will become apparent, to those ordinarily skilled in the art, upon review of the following description of the specific embodiments of the invention.

### BRIEF DESCRIPTION OF THE DRAWINGS

For a better understanding of the present invention, and to show more clearly how it may be carried into effect, reference will now be made, by way of example, to the accompanying drawings, which illustrate aspects of embodiments of the present invention and in which:

FIG. 1 is a very specific example of a surgery validation card provided in accordance with aspects of the invention;

FIG. 2A is an exploded perspective view of a surgery validation card with an instrument tray provided in accordance with aspects of the invention;

FIG. 2B is a perspective view of a surgery validation card integrated with a sleeve provided in accordance with aspects of the invention;

FIG. 3 is a flow chart illustrating the general method steps of a first method for performing and documenting a surgical pause provided in accordance with aspects of the invention;

FIG. 4 is a flow chart illustrating the general method steps of a second method for performing and documenting a surgical pause provided in accordance with aspects of the invention; and

FIG. 5 is a schematic diagram of a system for enabling the use a surgical validation card including machine-readable information provided in accordance with aspects of the invention.

### DETAILED DESCRIPTION OF THE INVENTION

A surgical pause is a well intentioned pre-surgery procedure designed to reduce the likelihood that a patient may undergo an incorrect surgical procedure. A problem is that the surgical pause is often not taken as seriously as it should be. In many cases, the health-care professionals involved assume that they are in the correct operating theater with the correct patient. So, for example, after a brief repetition of the surgical procedure to be performed is spoken aloud, the doctors and nurses (and other specialists) proceed under the assumption that they are performing the correct surgical procedure without further thought. Moreover, given the ad hoc way in which the surgical pause is carried out, there is no way to enforce compliance with the spirit of the surgical pause and/or to check after the surgery that the surgical pause was performed with the requisite care and diligence by the health-care professionals in the operating theater.

Health-care professionals, especially operating-room staff, are naturally cautious and risk averse when contemplating the adoption of new operating procedures and/or electronic systems that involve significant changes to their accepted procedures. Despite this, the aforementioned mistakes made in an operating theater can be attributed to not having a more systematic method for performing a surgical pause before a surgical procedure begins.

In accordance with aspects of the invention apparatus, systems and methods are provided that may enable compliance to the intended spirit of the surgical pause and that may provide a record that a surgical pause was performed with the requisite care and diligence by the operating-room staff. More specifically, some embodiments of the invention include a sterile surgery validation card suitable for use within a sterile field in an operating theater. The sterilized surgery validation card is then filled out and signed by the members of the operating-room staff within the sterile field (e.g. surgeons, anesthesiologists, nurses and the like) before the first incision and/or puncture of the surgical procedures occurs. In some embodiments, the surgery validation card includes without limitation a checklist for giving the operating-room staff a chance to double-check that the patient before them is the correct patient and to double-check that they are aware of what surgical procedure they are about to perform and on which body part of the patient. In even more specific embodiments, a surgery validation card includes a signature field in which one or more of the operating-room staff (e.g. including the surgeon, anesthesiologists and circulating nurse) sign to remind the staff of their responsibility to have performed the surgical pause with the requisite care and diligence. As such, the completed surgery validation card provides a record that the surgical pause was completed diligently and the completed surgery validation card can then be placed in the patient chart for future reference.

Additionally and/ore alternatively, some aspects of the invention provide a surgery validation card that can be integrated into a sterile sleeve in which either an instrument tray or patient body part may be inserted. Then before a surgical procedure begins the operating-room staff is forced to at least recognize the surgery validation card and remove it to begin the surgery, at which time they may be compelled to complete the surgery validation card.

Additionally and/or alternatively, some aspects of the invention provide a surgery validation card with machine-readable information such as may be provided in bar-codes and/or Radio Frequency Identification (RFID) tags. Further in accordance with even more specific aspects of the invention provided are electronically-aided systems and methods, which make use of the machine-readable information, for validating that a patient and surgical procedure to be performed are correct.

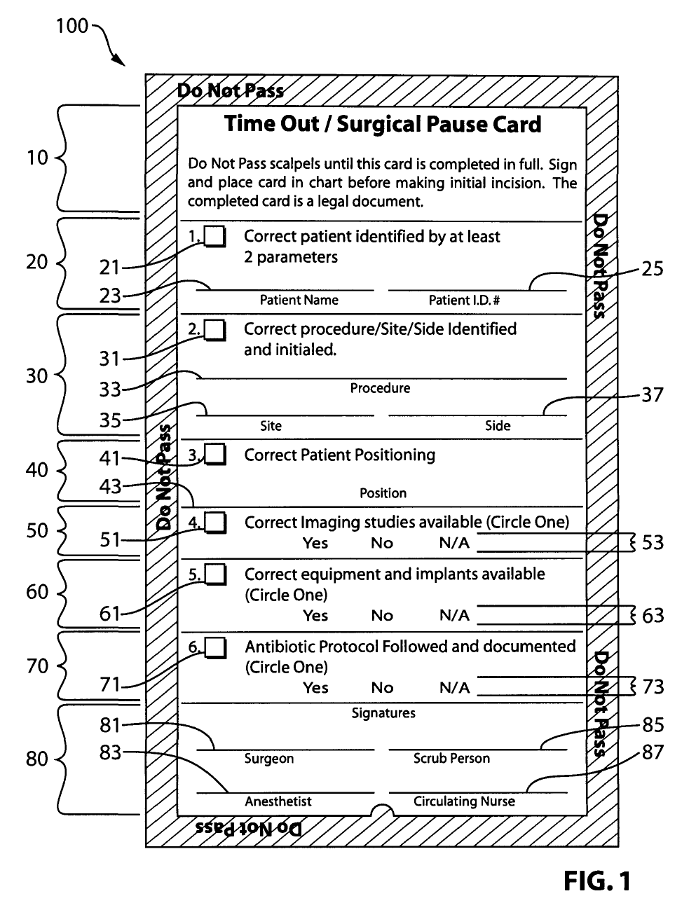

Referring to FIG. 1, shown is an example of a very specific example of a surgery validation card 100 provided in accordance with aspects of the invention. The surface of the surgery validation card 100 is a planar writing surface suitable for writing on with a pen or pencil, such as may be provided by, without limitation, paper, card-stock and label-stock. The surgery validation card 100 includes at least one field printed on the planar writing surface having a checklist to be completed for verifying a respective identity of a patient in an operating theatre and a surgical procedure to be performed on the patient. In some embodiments the checklist is divided into a number of fields. Each of the fields requires that the operating-room staff consider and verify a separate aspect of the impending surgical procedure. A completed surgery validation card 100 can then be removed from the sterile field and placed in the patient chart to serve as a record of the surgical pause. The fact that a written record is created for the surgical pause by employing the surgery validation card 100 may compel the operating-room staff to take the surgical pause more seriously and thereby adhere to the spirit of the surgical pause procedure.

In the very specific example shown in FIG. 1 the checklist on the surgery validation card 100 includes a title field 10, a patient identification field 20, a procedure identification field 30, a position verification field 40, an imaging check field 50, an equipment/implant check field 60, an antibiotic check field 70 and a signature field 80.

The title field 10 includes two subfields containing a document identifier 11 and a short list of instructions 13, respectively. The patient identification field 20 includes two respective sub-fields 23 and 25 for writing a patient name and corresponding patient identification number. The procedure identification field 30 includes three respective sub-fields 33, 35 and 37 for writing a respective surgical procedure name, a site name, and side. The position verification field 40 includes a single sub-field 43 for a respective position description for the corresponding surgical procedure name 33.

The imaging check field 50 includes a single sub-field 53 having a multiple-choice query to determine if any required imaging information is required for the surgical procedure. The equipment/implant check field 60 includes a single sub-field 63 having a multiple-choice query to determine if any required equipment/implants are required for the surgical procedure. The antibiotic protocol check field 70 includes a single sub-field 73 having a multiple-choice query to determine if a corresponding antibiotic protocol has been followed and documented for the surgical procedure.

The signature field 80 includes four signature sub-fields 81, 83, 85 and 87 for the signatures of the surgeon, anesthesiologist, scrub person and circulating nurse, respectively. In general, the signature field 80 has at least one sub-field for a signature of one of the health-care professionals in an operating theatre present during a surgical pause. In use, after the surgery validation card 100 has been filled out, each of the members of the operating-room staff included in the signature field should sign the surgery validation card 100 in the correct field to provide proof that the surgical pause was carried out with requisite care and diligence. Moreover, the signatures may help enforce to the signatories that they should be have taken the surgical pause seriously because executing the surgery validation card 100 involves a legal and professional responsibility.

Additionally and/or alternatively, each field 20, 30, 40, 50, 60 and 70 has a corresponding check-box 21, 31, 41, 51, 61 and 71 that is to be checked when the respective field is completed. In use, for example, the member of the operating room-staff that checks the boxes 21, 31, 41, 51, 61 and 71 can be different from the member of the operating-room staff that filled out the checklist embodied in the fields 20, 30, 40, 50, 60 and 70.

Additionally and/or alternatively, the surgery validation card 100 also includes machine-readable information (not shown in FIG. 1) storing information about a patient. In some embodiments, the machine-readable information includes at least one of a patient identifier, a surgical procedure identifier, a required equipment/implant listing, an allergy notice, a required image listing and antibiotic protocol reminder. In some embodiments the machine-readable information is stored in at least one of a bar-code printed on the surgery validation card and a Radio Frequency Identification (RFID) tag included on the surgery validation card.

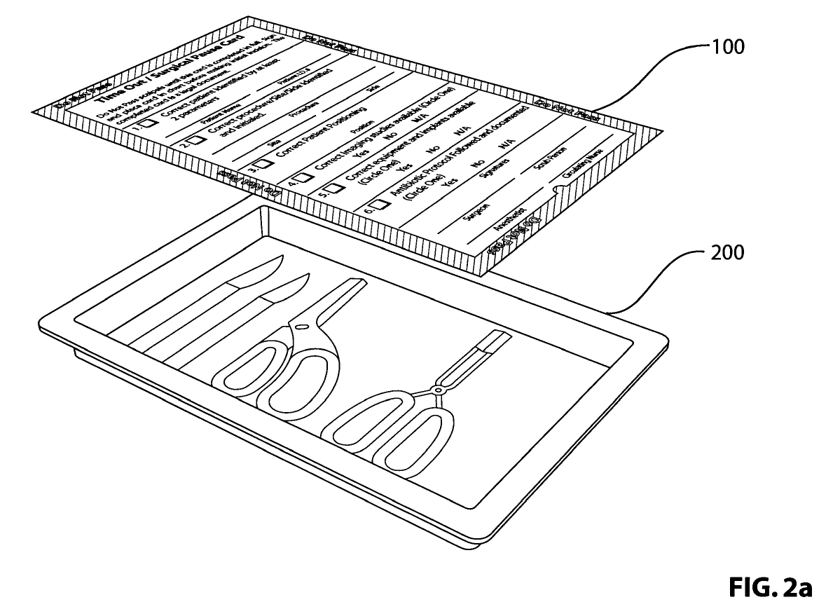

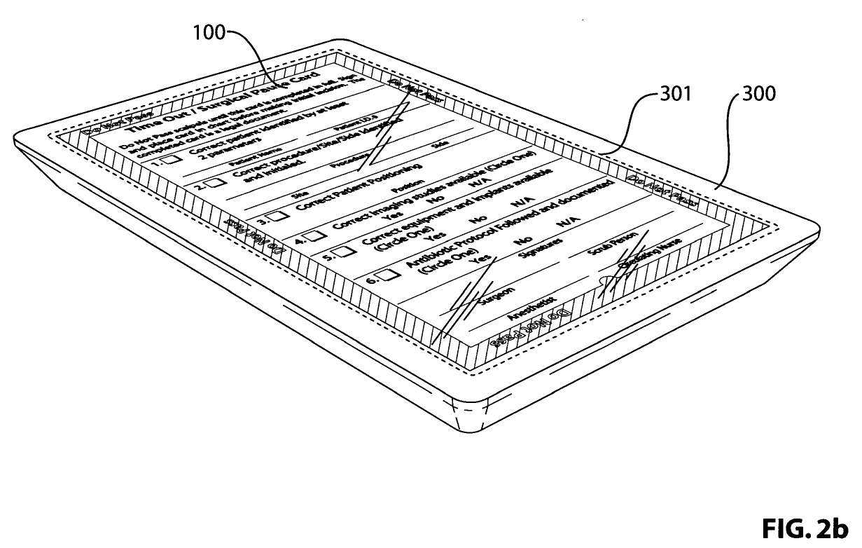

Turning to FIG. 2A, shown is an exploded perspective view of the surgery validation card 100 with an instrument tray 200 in accordance with aspects of the invention. In order for both the surgery validation card 100 and instrument tray 200 to be suitable for use in a sterile field in an operating theater, both must be sterilized. The surgery validation card 100 is preferably sterilized using gamma radiation and packaged outside a hospital. Alternatively, the surgery validation card 100 can be sterilized within the hospital. As shown in FIG. 2A, one use of the surgery validation card 100 is to place it over an instrument tray 200, so that the operating-room staff are forced to remove it before they can access the instruments 220 on the instrument tray 200. In this way, the operating-room staff are reminded to perform the surgical pause as mandated on the surgery validation card 100.

In another example, FIG. 2B shows a perspective view of the surgery validation card 100 integrated with a sleeve 300 in accordance with aspects of the invention. More specifically, the surgery validation card 100 is removably integrated with the sleeve 300. In this particular example, the surgery validation card 100 is connected to the sleeve 300 along a common perforated edge 301. In other embodiments, a surgery validation card is connected to the sleeve using a number of methods and/or appliances including, without limitation, adhesive tape, staples, clips, snaps, Velcro and the like. In use, an instrument tray or body part of the patient can be placed in the sleeve 300. If a body part is placed in the sleeve 300, the sleeve is preferably placed over the initial incision and/or puncture point of the surgical procedure to be performed.

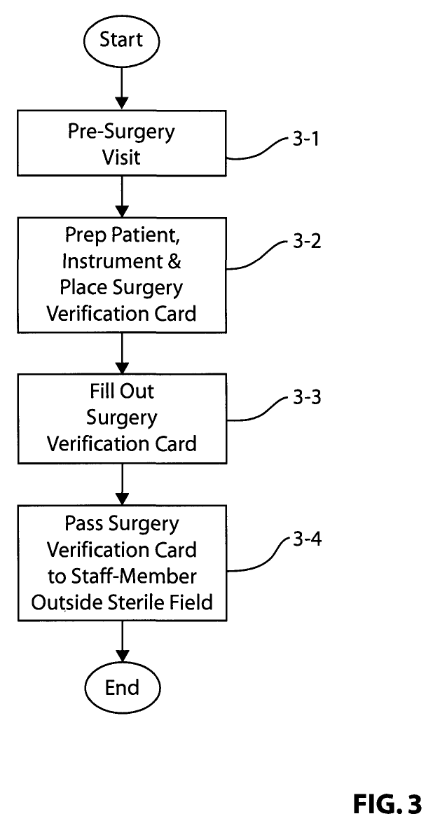

FIG. 3 is a flow chart illustrating the general method steps of a first method for performing and documenting a surgical pause in accordance with aspects of the invention. Starting with step 3-1, the first method includes a pre-surgery visit by the surgeon to the patient during which time the surgeon performs a pre-surgery consult and marks the initial incision point of the surgical procedure with a washable pen/marker. At step 3-2 the first method includes prepping the patient and the instruments in the operating theater along with arranging a surgery validation card within the sterile field. The surgery validation card may, without limitation, be arranged so that it is covering at least one of an instrument tray and a portion of the patient. In some cases the surgery validation card may also be provided with a sterilized and disposable permanent non-bleeding pen for marking and completing the checklist on the surgery validation card.

At step 3-3 the first method includes completing the surgery validation card before the first incision and/or puncture of the surgery begins. Once the surgery validation card is completed, it may be passed out of the sterile field by one of the operating-room staff to a staff member outside of the sterile field as indicated at step 3-4. Once a completed surgery validation card is outside the sterile field it can be inserted into a respective patient chart, which is typically not sterilized.

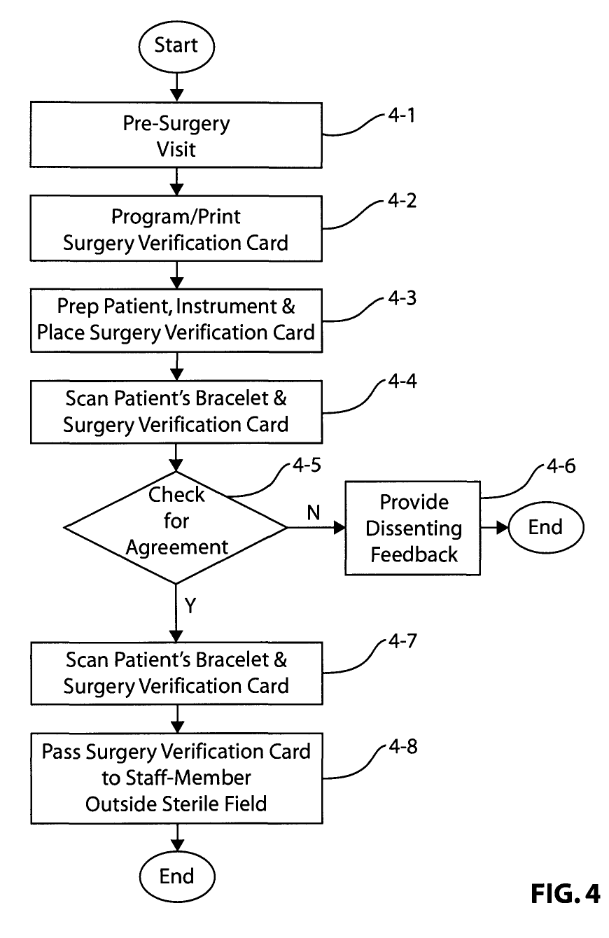

The first method can be modified to include the use of machine-readable information such as the type stored in a bar-code or a RFID tag. FIG. 4 shows a flow chart illustrating the general method steps of a second method for performing and documenting a surgical pause in accordance with aspects of the invention that includes the use of machine-readable information. Starting at step 4-1, the second method includes a pre-surgery visit by the surgeon to the patient during which the surgeon performs a pre-surgery consult and marks the initial incision point of the surgical procedure with a washable pen/marker. At step 4-2 the second method includes printing and/or programming the machine-readable information onto a surgery validation card, depending on whether or not a bar-code or RFID tag or a combination of both is used to store the machine-readable information. It may also be necessary to re-sterilize the surgery validation card if the programming and/or printing is done in a non-sterile environment. Step 4-3 includes prepping the patient and the instruments in the operating theater along with arranging a surgery validation card within the sterile field. The surgery validation card may, without limitation, be arranged so that it is covering at least one of an instrument tray and a portion of the patient. In some cases the surgery validation card may also be provided with a sterilized and disposable permanent non-bleeding pen for marking and completing the checklist on the surgery validation card.

Step 4-4 of the second method includes scanning machine-readable information on a surgery validation card and machine readable information on a patient bracelet. Subsequently, step 4-5 includes comparing the scanned machine-readable information from the surgery validation card and the patient bracelet. If the scanned machine-readable information from the surgery validation card and the patient bracelet do not agree (no path, step 4-5), dissenting feedback is provided at step 4-6 and the method ends. At such a point, the operating-room staff may be compelled to double-check the present situation and make a decision as to whether or not they should continue with the surgical procedure or abort the surgical procedure. If the scanned machine-readable information from the surgery validation card and the patient bracelet agree (yes path, step 4-5), affirmative feedback is provided, thereby indicating to the operating-room staff that at the very least the scanned information on the surgery validation card and that on the patient bracelet agree.

Following affirmative feedback from step 4-5, at step 4-7 the second method includes completing the surgery validation card before the first incision and/or puncture of the surgery begins. Once the surgery validation card is completed, it may be passed out of the sterile field by one of the operating-room staff to a staff member outside of the sterile field as indicated at step 4-8. Once a completed surgery validation card is outside the sterile field it can be inserted into a respective patient chart, which is typically not sterilized.

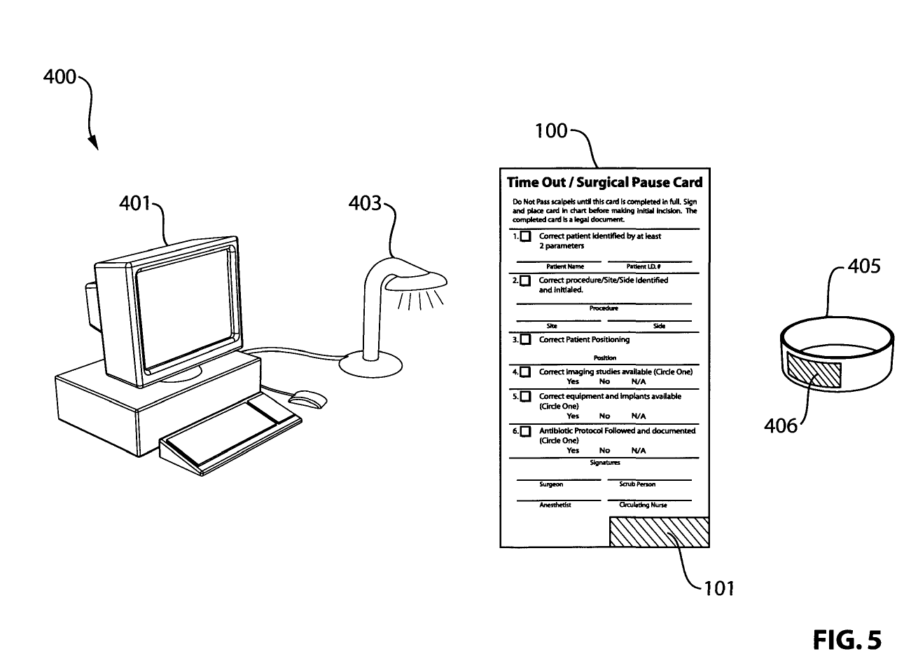

FIG. 5 is a schematic diagram of a system 400 for enabling the use a surgical validation card provided with machine-readable information provided in accordance with aspects of the invention. The system 400 includes workstation computer 401 (e.g. a personal computer, network computer, etc.), and a machine-scanner 403 coupled to the workstation computer 401. The workstation computer 401 includes a processor and a memory that is accessible by the processor. Those skilled in the art will appreciate that the workstation computer 401 also includes an additional suitable combination of hardware, software and firmware, and that the functional elements illustrated in FIG. 5 have only been provided to describe aspects of a very specific embodiment of the invention.

In some embodiments the machine-scanner 403 is suitable to scan information from bar-codes. Additionally and/or alternatively, the machine-scanner 403 is suitable to scan information from RFID tags.

Also shown in FIG. 5, for the sake of example only, are a patient bracelet 405 and a surgery validation card 100. The patient bracelet 405 has a label 406 with machine-readable information, as described above. Similarly, the surgery validation card 100 also has a label 101 with machine-readable information.

In operation, the system 400 is used to scan the labels 406 and 101 to verify whether or not the information on each is the same. The machine-scanner 403 is used to scan each label 406 and 101 individually and provide the information to the workstation computer 401. The workstation computer 401 upon receiving the scanned information on both labels compares the scanned information and provides feedback to a user. If the labels are the same, the feedback is affirmative. On the other hand if the information is not the same the feedback is dissenting and the operating-room staff are instructed to at least re-consider performing the surgical procedure.

Moreover, by scanning the labels 406 and 101 and storing the result of the comparison a record is created that can be stored in the memory of the workstation computer 401. In a hospital or another health-care facility, records of all surgical pauses can be created in this way. Each such record can be reviewed at a later time to verify that the individual(s) responsible for performing the surgical pauses were being diligent. That is, in accordance with aspects of the invention, the record created by scanning labels on a surgery validation card and a patient bracelet can be used as a metric for quality control and culpability.

A number of records created by the system can be stored in a database. To that end, aspects of the invention may be embodied in a number of forms. For example, various aspects of the invention can be embodied in a suitable combination of hardware, software and firmware. In particular, some embodiments include, without limitation, entirely hardware, entirely software, entirely firmware or some suitable combination of hardware, software and firmware. In a preferred embodiment, the invention is implemented in software, which includes but is not limited to firmware, resident software, microcode, etc.

Additionally and/or alternatively, aspects of the invention can be embodied in the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

A computer-readable medium can be an electronic, magnetic, optical, electromagnetic, infrared, or semiconductor system (or apparatus or device) or a propagation medium. Examples of a computer-readable medium include a semiconductor and/or solid-state memory, magnetic tape, a removable computer diskette, a random access memory (RAM), a read-only memory (ROM), a rigid magnetic disk and an optical disk. Current examples of optical disks include, without limitation, compact disk-read only memory (CD-ROM), compact disk-read/write (CD-RW) and DVD.

In accordance with aspects of the invention, a data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (i.e. I/O devices)—including but not limited to keyboards, displays, pointing devices, etc.—can be coupled to the system either directly or through intervening I/O controllers.

Network adapters may also be coupled to the system to enable communication between multiple data processing systems, remote printers, or storage devices through intervening private or public networks. Modems, cable modems and Ethernet cards are just a few of the currently available types of network adapters.

While the above description provides example embodiments, it will be appreciated that the present invention is susceptible to modification and change without departing from the fair meaning and scope of the accompanying claims. Accordingly, what has been described is merely illustrative of the application of aspects of embodiments of the invention and numerous modifications and variations of the present invention are possible in light of the above disclosure.

I claim:

1. A surgery validation card comprising:
a planar writing surface;
at least one field printed on the planar writing surface having a checklist to be completed for verifying a respective identity of a patient in an operating theatre and a surgical procedure to be performed on the patient.

2. A surgery validation card according to claim 1 that is sufficiently sterilized so as to be suitable for use in a sterile field within an operating theatre.

3. A surgery validation card according to claim 1 removably integrated into a sleeve.

4. A surgery validation card according to claim 3 wherein the surgery validation card is connected to the sleeve employing at least one of a perforated edge, a connecter and adhesive tape.

5. A surgery validation card according to claim 1 further comprising machine-readable information storing information about the patient.

6. A surgery validation card according to claim 5 wherein the machine-readable information includes at least one of a patient identifier, a surgical procedure identifier, a required equipment/implant listing, an allergy notice, an required image listing and antibiotic protocol reminder.

7. A surgery validation card according to claim 5 wherein the machine-readable information is stored in at least one of a bar-code printed on the surgery validation card and an Radio Frequency Identification (RFID) tag included on the surgery validation card.

8. A surgery validation card according to claim 1 further comprising a title field printed on the planar writing surface including a document identifier and a short list of instructions.

9. A surgery validation card according to claim 1 further comprising a patient identification field, printed on the planar writing surface, including sub-fields for writing a respective patient name and corresponding patient identification number.

10. A surgery validation card according to claim 1 further comprising a procedure identification field, printed on the planar writing surface, including sub-fields for writing a respective surgical procedure name, a site name, and side.

11. A surgery validation card according to claim 10 further comprising a position verification field, printed on the planar writing surface, including a sub-field for writing a respective position description for the corresponding surgical procedure name.

12. A surgery validation card according to claim 1 further comprising an imaging check field, printed on the planar writing surface, including a sub-field having a multiple-choice query to determine if any required imaging information is required for the surgical procedure.

13. A surgery validation card according to claim 1 further comprising an equipment/implant check field, printed on the planar writing surface, including a sub-field having a multiple-choice query to determine if any required equipment/implants are required for the surgical procedure.

14. A surgery validation card according to claim 1 further comprising an antibiotic protocol check field, printed on the planar writing surface, including a sub-field having a multiple-choice query to determine if a corresponding antibiotic protocol has been followed and documented for the surgical procedure.

15. A surgery validation card according to claim 1 further comprising a signature field, printed on the planar writing surface, including at least one sub-field for a signature of one of the health-care professionals in an operating theatre present during a surgical pause.

16. A method of performing a surgical pause comprising:
providing a sterile surgery validation card within a sterile field of an operating theatre;
completing the surgery validation card before the initial incision or puncture of a surgical procedure to verify at least the identity of a patient and the surgical procedure to be performed; and
passing the completed surgery validation card outside the sterile field so that the completed surgery validation card can be placed into a corresponding patient chart.

17. A method according to claim 16 further comprising:
scanning machine-readable information on a surgery validation card and machine readable information on a patient bracelet;
comparing the scanned machine-readable information from the surgery validation card and the patient bracelet;
providing affirmative feedback if the surgery validation card and patient bracelet have the same scanned machine-readable information, and thereby by permitting the surgery validation card to be completed; and
providing dissenting feedback if the surgery validation card and patient bracelet do not have the same scanned machine-readable information.

18. A method according to claim 16, wherein providing the sterile surgery validation card includes placing the incomplete surgery validation card on one of the surgical instruments or on a portion of the patient.

19. A method according to claim 16 wherein passing the completed surgery validation card outside the sterile field includes physically separating the surgery validation card from a sleeve.

20. A surgery validation system comprising:
a surgery validation card for verifying at least the identity of a patient and a surgical procedure to be performed, the surgery validation card including machine-readable information storing at least the identity of a patient and the name of the surgical procedure to be performed on the patient;
a patient bracelet including machine-readable information storing at least the identity of a patient and the name of the surgical procedure to be performed on the patient;
a machine-scanner for scanning machine-readable information and providing a scanned output; and
a workstation computer connectable to the machine-scanner for receiving the scanned output from the machine-scanner.

21. A system according to claim 20, wherein the machine-scanner and workstation computer are integral with one another.

22. A system according to claim 20, wherein the machine-readable information on the surgery validation card is stored in at least one of a bar-code printed on the surgery validation card and an Radio Frequency Identification (RFID) tag included on the surgery validation card.

23. A system according to claim 20, wherein the machine-readable information on the patient bracelet is stored in at least one of a bar-code printed on the surgery validation card and an Radio Frequency Identification (RFID) tag included on the surgery validation card.

24. A system according to claim 20, wherein the workstation computer includes a computer usable program code including program instructions for:
receiving scanned machine-readable information from the surgery validation card and the patient bracelet;
comparing the scanned machine-readable information from the surgery validation card and the patient bracelet;
providing affirmative feedback if the surgery validation card and the patient bracelet have the same scanned machine-readable information; and
providing dissenting feedback if the surgery validation card and the patient bracelet do not have the same scanned machine-readable information.

25. A system according to claim 24, wherein the computer usable program code further includes program instructions for creating a record of the comparison.

26. A system according to claim 25, wherein the computer usable program code including program instructions for updating a database of records, wherein each record contains the results and a particular comparison.
